(19) United States
(12) Patent Application Publication
(10) Pub. No.: US 2014/0088050 A1
(43) Pub. Date: Mar. 27, 2014

(54) USE OF ANDROGEN FOR DISEASE TREATMENT

(71) Applicant: Edward M. Lichten, Birmingham, MI (US)

(72) Inventor: Edward M. Lichten, Birmingham, MI (US)

(21) Appl. No.: 14/035,237

(22) Filed: Sep. 24, 2013

Related U.S. Application Data
(60) Provisional application No. 61/704,797, filed on Sep. 24, 2012.

(51) Int. Cl. A61K 31/585; A61K 31/567; A61K 31/565; A61K 31/58; A61K 31/5685; A61K 31/568

(52) U.S. Cl. CPC A61K 31/585; A61K 31/5685; A61K 31/568; A61K 31/565; A61K 31/58; A61K 31/567
USPC 514/170

(57) ABSTRACT

By lowering and then after raising the amount of SHBG in the body, an effective method for treatment of various diseases is achieved. First and seconds androgens, namely, anabolic steroids are used to both lower the SHBG and, thereafter raise the testosterone in the body until equilibrium between the SHBG and the second steroid, to normalize the FAI. Stanozolol is the preferred anabolic steroid used herein to lower the SHBG and testosterone is used to normalize the FAI.

### CROSS-REFERENCE TO RELATED APPLICATION

This application is a completion application of co-pending U.S. Provisional Patent Application Ser. No. 61/704,797 filed Sep. 24, 2012, the entire disclosure of which is hereby incorporated by reference.

### BACKGROUND OF THE INVENTION

1. Field of the Invention

The present invention pertains to means and methods for treating various diseases. More particularly, the present invention pertains to means and methods for overcoming exposure to environmental toxins. Even more particularly, the present invention pertains to the use of androgens for lowering the level of SHBG (Sexual Hormone Binding Globulin) in a patient.

2. Description of the Prior Art

As to is known to those skilled in the art to which the present invention pertains, there has been reported the use of an androgen, such as stanozolol, for blocking liver production of SHBG. Further, the prior art is replete with reports showing the use of the laboratory measurement of SHBG using an androgen as a marker; sensitivity test, androgen receptor, mutation detection; and so forth. Furthermore, U.S. Pat. Nos. 7,186,706; 6,583,129 and 6,139,873 further illustrates the utilization of stanozolol and other androgens in connection with SHBG testing.

Heretofore, the cause of disease has been the subject of many hypotheses, including organic; genetic; environmental and so forth.

As is disclosed hereinafter, the predicate for the present invention resides in the postulate that environmental toxins cause disease and these toxins affect the body only through the levels of SHBG. Therefore, by blocking or lowering the level of SHBG, the equilibrium of the body is changed, reducing the effects of environmental toxins.

In essence, by utilizing an androgen to lower the SHBG in the body, there is a decrease in the effect of the endocrine disruption compounds, thereby reducing the propensity of the body to be diseased from the endocrine disrupting compounds.

### SUMMARY OF THE INVENTION

In accordance with the present invention, there is provided both means and a method for treatment of disease by lowering the SHBG in the body through the utilization of an androgen and, thereafter raising the testosterone in the body through the use of a second androgen to bring the FAI (Free Androgen Index) to acceptable levels. More specifically, by utilizing selected amounts of a first and second androgen, it is possible to first lower the amount of SHBG in the body with the first androgen and, once the reduced level is achieved, raising the FAI in the body with another androgen to achieve equilibrium between the SHBG and the androgen.

Preferably, the androgens used herein are steroids. Both naturally occurring and synthetic steroids may be used.

For a more complete understanding of the present invention, reference is made to the following detailed description of the invention.

### DESCRIPTION OF THE PREFERRED EMBODIMENT

As above-noted the present invention provides both means and a method for treatment of disease by first lowering the SHBG in the body through the utilization of a first androgen and, thereafter, once the SHBG reaches the desired reduced level utilizing a second androgen to raise the FAI to an acceptable FAI level.

More specifically, by utilizing selected amounts of a first androgen, it is possible to lower the amount of SHBG in the body.

Generally, the androgen is administered over a period from about four to eight weeks in order to lower the SHBG to about 30 to about 45 nanomoles per liter (nmol/l) for a female and to about 5 to 15 nmol/l for a male.

After the treatment with the first androgen to lower the SHBG, it is necessary to then add an androgen back into the system to raise the serum testosterone levels back to normal levels.

By first lowering the amount of SHBG with the androgen and, then, adding an androgen back into the body, the effective treatment of diseases caused by environmental toxins is substantially increased.

In practicing the present invention in lowering the SHBG both synthetic and natural androgens may be used. Representative natural androgens which may be used and include, for example, dihydroepiandrosterone; androstenedione; androstenediol; androsterone; dihydrotestosterone; testosterone; and nandrolone.

Synthetic androgens, to lower the SHBG such as stanozolol, oxandrin, furzabolin, dianabol, danazol, anadrol and the like may be used. It is possible, although not preferred, to use a combination of synthetic and natural androgens for lowering the SHBG.

Preferably, the androgen used to lower the SHBG in the practice hereof is an anabolic steroid. Anabolic steroids are well known and commercially available.

Among the preferable and useful anabolic steroids for SHBG lowering in accordance herewith are stanozolol, dianabol, danazol and anadrol.

Most preferably, the steroid stanozolol is used for lowering the SHBG. The stanozolol can be administered either orally or as an injectible.

The gender of the patient or person determines the amount of steroid necessary to lower the SHBG in order to achieve the desired Free Androgen Index (FAI). For a male, the Free Androgen Index should range from about 1 to about 2. For a female the FAI should range from about 0.03 to about 0.10. Thus, a male needs to have an SHBG level of from about 5 to about 15 (nmol/l) and a female from about 30 nmol/l to about 45 nmol/l in order to ultimately achieve the desired FAI.

Regardless of gender, generally, from about 4.0 milligrams to about 8.0 milligrams of the stanozolol is administered orally, per day, for about four to about eight weeks, in order to effectuate the lowering of the SHBG.

Where injections are used, generally, from about 10.0 mgs to about 50.0 mg are injected once per week for about four to eight weeks.

As noted, after the SHBG has been reduced to an acceptable level a second steroid is then administered back into the body to rebalance the equilibrium in the body between the SHBG and the second androgen. Preferably, the second steroid is testosterone.

Depending on the gender of the patient, the amount of testosterone added back into the body will vary.

Where the patient is a male, generally from about 100 mgs to about 250 mgs of “added back” testosterone is administered, once a week, on a weekly basis, as an injection for about four to about eight weeks.

With a female patient the amount of testosterone administered will generally range from about 15 to about 70 milligrams per week for about four to about eight weeks.

The protocol may be continued indefinitely.

By following the protocol herein described, generally, in about four to eight weeks, equilibrium between the SHBG and the testosterone is achieved and the FAI will range within the level described above. Furthermore, by using the levels of androgen, described above, there is a concomitant “re-balancing” of appropriate testosterone levels with a decrease in SHBG.

Having, thus, described the invention, what is claimed is:

1. A method for lowering the SHBG in a person, comprising:
a) administering a predetermined amount of a first androgen to a person over a period of from about four to eight weeks to lower the SHBG in the person's body to a pre-selected level, and
b) thereafter, administering a second androgen into the person's body for a period of about four to about eight weeks to achieve an acceptable Free Androgen Index according to the gender of the person.

2. The method of claim 1 wherein the first and second androgens are steroids.

3. The method of claim 2 wherein the first steroid is selected from the group of consisting of dihydroepiandrosterone; androstenedione; androstenediol;
androsterone; dihydrotestosterone; testosterone and nandrolone stanozolol, oxandrin, furazabol, dioxadol, danazol and anadrol.

4. The method of claim 2 wherein the second steroid is selected from the group consisting of testosterone and nandrolone.

5. The method of claim 4 wherein the first steroid is stanozolol.

6. The method of claim 2 wherein the first steroid is administered orally in an amount ranging from about 4.0 milligrams to about 8.0 milligrams daily for about four to eight weeks to lower the SHBG.

7. The method of claim 6 wherein the person is male and the second steroid is administered, as an injection, in an amount ranging from about 100 milligrams to about 250 milligrams, once a week, for about four to about eight weeks.

8. The method of claim 6 wherein the person is a female and the added back steroid is administered once a week, as an injection in an amount which ranges from about 15 milligrams to about 70 milligrams.

9. The method of claim 1 wherein the SHBG is lowered to about 5 to 15 nanomoles per liter for a male and to about 30 to 45 nanomoles per liter for a female.

10. The method of claim 1 wherein the Free Androgen Index after treatment ranges from about 1 to about 2 for a male and from about 0.03 to about 0.10 for a female.
